(19) United States
(12) Patent Application Publication
(10) Pub. No.: US 2026/0000815 A1
(43) Pub. Date: Jan. 1, 2026

(54) GUIDED BONE REGENERATION MATERIAL, METHOD OF MAKING AND USES THEREOF

(71) Applicant: The University of Hong Kong, Hong Kong (HK)

(72) Inventors: Hai Ming WONG, Hong Kong (HK); Quan Li LI, Shenzhen (CN); Le ZHANG, Hong Kong (HK)

(21) Appl. No.: 19/243,761

(22) Filed: Jun. 20, 2025

Related U.S. Application Data
(60) Provisional application No. 63/664,789, filed on Jun. 27, 2024.

(51) Int. Cl. A61L 31/02; A61L 31/04; A61L 31/14; A61L 31/16; C08L 33/02

(52) U.S. Cl. CPC A61L 31/028; A61L 31/048; A61L 31/146; A61L 31/16; C08L 33/02; A61L 2300/406; A61L 2430/02

(57) ABSTRACT

The present invention provides a guided bone regeneration (GBR) material with antibacterial properties. The GBR material is a multi-component material, including an eggshell membrane (ESM) which is functionalized by mineralization in the presence of low-molecular-weight polyacrylic acid (LPAA). Thus, the ESM therefore includes at least one mineral deposited therein, preferably, intrafibrillar deposition, for example apatite and effective amounts of LPAA. The mineralized GBR material is preferably porous, and has good mechanical properties. A method of making mineralized GBR material which preferably contains intrafibrillar mineralization is also provided.

### CROSS-REFERENCE TO RELATED APPLICATIONS

The present application claims the priorities from the U.S. provisional patent application Ser. No. 63/664,789 filed Jun. 27, 2024, and the disclosure of which is incorporated herein by reference in its entirety.

### FIELD OF THE INVENTION

The invention relates to an intrafibrillar mineralized eggshell membrane (ESM) for bone regeneration, specifically in the field of dentistry.

### BACKGROUND OF THE INVENTION

Loss of oral bone can be caused by a variety of pathologies, such as osteoporosis, tumors, infections, inflammation, trauma, and tooth extraction. The conditions are causing an increasing number of bone defects worldwide. Loss of alveolar bone can be clinically managed by promoting bone growth with guided bone regeneration (GBR). GBR technique is a surgical approach for bone tissue regeneration that prevents fast-growing fibroblast and connective tissues from migrating into the damaged area by using a space-maintaining barrier membrane to allow enough time for the migration, proliferation, and mature of bone cells (FIG. 1). The GBR uses a membrane to support alveolar bone regeneration at a bony defect and is becoming popular as a solution to address insufficient bone volume.

An ideal GBR membrane must fulfill several key requirements. It must protect and stabilize cells, facilitate the proliferation and differentiation of mesenchymal stem cells (MSCs), and maintain the integrity and mechanical strength of the structure until new bone is formed. Additionally, the membrane should be able to deliver bioactive agents and inhibit or kill bacteria. However, finding a material that satisfies all of these criteria has proven difficult. While some multi-component materials, including those incorporating antibiotics, have been used in GBR, the increasing issue of antibiotic resistance poses a serious challenge to the long-term success of these approaches. Antibiotic-resistant bacteria can compromise the healing process and negatively affect post-surgical patient health, thus limiting the effectiveness of current GBR treatments.

Staphylococcus aureus (S. aureus) is one of the primary causes of soft tissue infections and often colonizes soft tissues in the oral cavity. Its presence can lead to significant clinical issues such as pain, swelling, inflammatory reactions, and even septicemia. The use of antibiotics such as ampicillin, kanamycin, and norfloxacin can induce mutations in S. aureus, resulting in resistance to a wide range of antibiotics, including tetracycline and chloramphenicol. This growing antibiotic resistance problem poses a major threat to the success of GBR procedures, as conventional antibiotic treatments may become ineffective. This highlights the need for antibacterial GBR materials capable of targeting S. aureus without relying on traditional antibiotics.

WO2022136667A1 discloses a GBR membrane incorporating apatite. While apatite is known for its bioactivity, the apatite in this reference is typically industrially prepared and added as a premixed component. This exogenous apatite lacks the fine control over crystal morphology and biological behavior observed in biomimetically mineralized structures. Specifically, such membranes fail to replicate bone-like intrafibrillar mineralization and do not enhance the mechanical properties of the membrane itself.

US20200179569A1 describes the use of linear polyacrylic acid (LPAA) as part of a bone implant surface coating; however, its function is merely structural, requiring surface coupling with diamine linkers to facilitate further functionalization. In that context, LPAA serves no intrinsic bifunctional role.

US20090022811A1 discloses mineralized collagen or polymer membranes exhibiting improved osteogenic and antibacterial properties by incorporating elements such as zinc phosphate. Although zinc provides antimicrobial effects, it lacks the capacity to promote osteoinduction and its antibacterial action is nonspecific and potentially cytotoxic at elevated concentrations.

Current GBR membranes often fail to meet all of the necessary functional requirements. While some membranes support osteogenesis, they typically lack effective antibacterial properties, and many do not maintain the required mechanical strength during the regeneration process. The challenge is further compounded by bacterial infections, particularly from S. aureus, which can disrupt bone healing and regeneration. Moreover, the reliance on antibiotic-based solutions in GBR raises significant concerns about the rise of antibiotic resistance, making it clear that alternative antimicrobial strategies are urgently needed.

### SUMMARY OF THE INVENTION

It is an object of the present invention to provide compositions for stimulating oral bone growth in a subject in need thereof. There is also a need for methods of stimulating oral bone growth in a subject in need thereof.

According to the present invention, a revolutionary strategy for accessing the intrafibrillar mineralization of ESM is provided. By adding LPAA into calcification solution, it achieves killing bacterium and simultaneously realizing cell proliferation. In accordance with the various embodiments of the present invention, a functionalized bioactive GBR membrane is designed by adding LPAA into the calcification solution to endow the antibacterial property of LPAA to the membrane by binding antibacterial LPAA to apatite while the apatite was intrafibrillar generated in the membrane fibers.

Unlike conventional use of LPAA as a surface modifier or inert component, in accordance with the embodiments of the present invention, LPAA actively induce intrafibrillar apatite formation and simultaneously confer antibacterial properties. This dual biofunctionality is critical to achieving both bone regeneration and infection control without relying on traditional antibiotics.

In one aspect, the present invention provides a guided bone regeneration material, which includes a mineralized eggshell membrane (ESM) comprising fibers and fibrils, intrafibrillarly deposited apatite and low-molecular-weight polyacrylic acid (LPAA) bound to the apatite. The intrafibrillarly deposited apatite comprises aligned, densely packed apatite crystals formed within the internal structures of the fibers and fibrils, such that a mineral phase is embedded inside a fibrillar matrix rather than externally coated onto the ESM, and is guided by the presence of LPAA. The use of intrafibrillar mineralization process mimics natural bone formation, enhancing both mechanical stiffness and bioactivity of the GBR membrane.

In one embodiment, the ESM is an outer shell membrane including mammillary knobs. The apatite crystals of the intrafibrillarly deposited apatite are fluorapatite exhibiting X-ray diffraction peaks at 20 angles of approximately 25.8°, 31.8°, 32.2°, 32.9°, and 53.1°. The LPAA has an average molecular weight of 1,000-20,000 Da. And the LPAA is present in an amount effective to inhibit bacterium growth.

In one embodiment, the bacterium comprises Staphylococcus aureus, Streptococcus mutans.

In another embodiment, the guided bone regeneration material has an average pore size between 250 nm to 1500 nm. Specifically, it is effective in preventing the infiltration of fast-growing fibroblasts and connective tissue cells into the defect site, which can otherwise outcompete bone-forming cells and inhibit proper bone regeneration.

In another embodiment, the ESM is mineralized for one day and has an average pore size between 800 nm to 1500 nm.

In yet another embodiment, the ESM is mineralized for three days and has an average pore size of between 300 nm to 600 nm.

In another embodiment, the guided bone regeneration material has a porosity of approximately 40% to 70%.

In yet another embodiment, the guided bone regeneration material has a Young's modulus of at least about 100 MPa and up to about 500 MPa.

In one embodiment, the fibers and fibrils are derived solely from the ESM and are free of exogenous polymeric fibers.

In another embodiment, the guided bone regeneration material is biocompatible with Sprague-Dawley bone marrow mesenchymal stem cells (SD-BMSCs).

In yet another embodiment, the guided bone regeneration material promotes ectopic and in situ osteogenesis.

In other aspect, the present invention provides a method for preparing the guided bone regeneration material. The method includes contacting an unmineralized ESM with a dopamine solution at a concentration of about 1 to 5 mg/mL for 24 hours to form a dopamine-treated ESM; immersing the dopamine-treated ESM in a calcification solution comprising LPAA at a concentration of about 10 to 15 mg/mL; and incubating at 37° C. for 12 to 24 hours to induce intrafibrillar mineralization to form the guided bone regeneration material. The LPAA guides formation of aligned, densely packed apatite crystals embedded within an internal fibrillar matrix. The guided bone regeneration material exhibits improved Young's modulus and porosity relative to the unmineralized ESM.

In one embodiment, the calcification solution includes 5.83 mmol/L CaCl₂·2H₂O, 3.5 mmol/L K₂HPO₄, 1.17 mmol/L NaF and 135.7 mmol/L NaCl, buffered with 10 wt % Tris and 1 mol/L HCl at pH 6.9.

In another embodiment, the incubation is performed at 37° C. for at least about 6 hours and less than about 72 hours.

In further aspect, the present invention provides a method for promoting bone regeneration in a subject in need thereof, comprising applying the guided bone regeneration material to a bone defect site in the subject.

In one embodiment, the subject suffers from periodontal disease in need of bone regeneration.

In another embodiment, the subject suffers from hyperglycemia or impaired wound healing.

In one embodiment, the guided bone regeneration material induces early-stage osteogenesis within 7 days and promotes defect closure within 3 months.

### BRIEF DESCRIPTION OF THE DRAWINGS

Embodiments of the invention are described in more details hereinafter with reference to the drawings, in which:

FIG. 1 is a schematic showing the mineralization process of outer eggshell membrane (ESM) and the bone-inductive mechanism of mineralized outer ESM. Left side is an isolation methodology, activation, and mineralization of outer ESM. Right side is the mineralized ESM promoted bone regeneration bICy preventing the invasion of connective tissues and by killing pathogenic bacterium;

FIG. 2A shows SEM micrograph showing the interface between the membrane and mammillary bodies (MB), with differentiation of the inner shell membrane (ISM) and outer shell membrane (OSM); scale bar: 20 μm;

FIG. 2B shows detail of the inner shell membrane (ISM) and the limiting membrane (LM); scale bar: 2 μm; and

FIG. 2C shows TEM micrograph of outer membrane fibers, highlighting the collagen core (C) and glycoproteic mantle (M), separated by extra-fiber spaces (E);

FIG. 3A is an image showing the surface of ESM fibers by scanning electron microscope (SEM);

FIG. 3B is an image showing cross-section of ESM fibers by transmission electron microscope (TEM); and

FIG. 3C is an image showing the surface of mineralized ESM fibers by SEM; and

FIG. 3D is an image showing cross-section of mineralized ESM fibers by TEM; scale bars: (FIG. 3A, FIG. 3C) 200 nm, (FIG. 3B, FIG. 3D) 500 nm;

FIG. 4A is an image showing the surface of intrafibrillar mineralized ESM on the first day;

FIG. 4B is an image showing cross-section of the intrafibrillar mineralized ESM on the first day;

FIG. 4C is an image showing the surface of extrafibrillar mineralized ESM on the third day; and

FIG. 4D is an image showing cross-section of extrafibrillar mineralized ESM on the third day; scale bars: (FIGS. 4A-4B) 2 μm, 200 nm (the inset), (FIG. 4C) 1 μm, (FIG. 4D) 2 μm;

FIG. 5A is a plot showing x-ray diffraction of mineralized ESM;

FIG. 5B is a plot showing young's modulus of unmineralized membrane and mineralized membrane at different stages;

FIG. 5C is a plot showing porosity and pore size of the unmineralized membrane and mineralized membrane at different stages; and

FIG. 5D is a plot showing thermogravimetric analysis of mineralized membrane at different stages;

FIG. 6 is a plot showing the absorbance of S. aureus in various concentrations of low-molecular-weight polyacrylic acid (LPAA) and S. aureus and MBC of LPAA at various concentrations of Staphylococcus aureus. Asterisk (*) indicates no colony formation on agar plates after incubation of S. aureus with LPAA at a certain concentration;

FIG. 7A shows no bacterial growth on the mineralized ESM by SEM;

FIG. 7B shows bacterial growth on the unmineralized ESM by SEM;

FIG. 7C is an image showing dead bacterium on the mineralized ESM by laser scanning confocal microscope (CLSM);

FIG. 7D is an image showing live bacteria on the unmineralized ESM by CLSM; scale bars: (FIGS. 7A-7B) 2 μm, (FIGS. 7C-7D) 100 μm;

FIG. 8A shows cells spread and proliferate on the mineralized ESM by SEM;

FIG. 8B shows cells spread and proliferate on the unmineralized ESM by SEM;

FIG. 8C shows cells spread and proliferate on the mineralized ESM by CLSM;

FIG. 8D shows cells spread and proliferate on the unmineralized ESM by CLSM. Scale bars: (FIGS. 8A-8B) 20 μm, (FIGS. 8C-8D) 100 μm;

FIG. 9A shows the in vitro osteogenic activity evaluation of mineralized outer ESM, outer ESM, and Bio-Gide®; BMSCs were isolated from samples by Transwell, and the potential of different materials to induce ectopic osteogenesis was assessed through alkaline phosphatase (ALP) staining and CN staining on days 7 and 14, respectively;

FIG. 9B shows the in vitro osteogenic activity evaluation of mineralized outer ESM, outer ESM, and Bio-Gide®; BMSCs were seeded on the sample surface, and the potential of different materials to induce in situ osteogenesis was observed through ALP and CN staining on days 7 and 14, respectively;

FIG. 9C shows the results of ALP staining and CN staining for BMSCs isolated by Transwell and seeded on mineralized outer ESM, outer ESM, and Bio-Gide® surfaces on days 7 and 14;

FIG. 9D shows the results of ALP staining and CN staining for BMSCs seeded directly on the surface of mineralized outer ESM, outer ESM, and Bio-Gide® on days 7 and 14; and

FIG. 10 shows the 3D reconstruction of the bone defect area. Mineralized outer ESM induces osteogenesis earlier than the other groups at the fourth week. A higher rate of closure of the defect area is observed in the mineralized outer ESM group by the third month.

### DETAILED DESCRIPTION

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

“Intrafibrillar mineralization” as used herein refers to mineralization (such as HA minerals) within the gap zone of fibrils.

“Extracellular mineralization” as used herein refers to mineralization (such as HA minerals) formed on the surface of fibrils.

“Intrafibrillarly deposited apatite” refers to apatite crystals that are formed and embedded within the internal structure of collagen-rich fibrils in the eggshell membrane, as opposed to apatite that is simply adsorbed or deposited on the external surface of the fibers. The crystals are aligned and densely packed along the axis of the fibrils, mimicking natural bone mineralization.

“Fibrils” refer to nanoscale substructures within the fibers of the eggshell membrane (ESM), typically composed of collagen. “Fibers” refer to larger, microscale structural units visible under SEM, comprising bundled fibrils and other matrix components.

“Low-molecular-weight polyacrylic acid (LPAA)” means a polyacrylic acid polymer having a weight-average molecular weight of less than 20,000 Da, preferably between 1,000 and 10,000 Da, and having carboxyl functional groups capable of inducing mineralization and exhibiting antibacterial activity.

“Porosity” refers to the total volume fraction of pores within the material, as measured by MIP method under standard conditions. Unless otherwise stated, it is expressed as a percentage of the total volume of the membrane.

“Average pore size” refers to the median diameter of pores within the mineralized eggshell membrane, as determined by MIP method. Reported values represent the midpoint pore diameter in a distribution curve.

“Ectopic osteogenesis” refers to bone formation induced at a non-skeletal site (e.g., subcutaneous or muscular tissue), usually assessed by in vitro culture or in vivo implantation models.

“In situ osteogenesis” refers to bone formation occurring directly on or within the implanted material at a bone defect site.

“Calcification solution” refers to a buffered aqueous solution comprising calcium and phosphate ions, optionally including fluoride and polyacids such as LPAA, and maintained at a pH and ionic strength conducive to apatite mineral formation.

Recitation of ranges of values herein are merely intended to serve as a shorthand method of referring individually to each separate value falling within the range, unless otherwise indicated herein, and each separate value is incorporated into the specification as if it were individually recited herein.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

The disclosed method and compositions may be understood more readily by reference to the following detailed description of particular embodiments and the Example included therein and to the Figures and their previous and following description.

Guided bone regeneration (GBR) is a surgical procedure that utilizes bone grafts with barrier membranes to reconstruct small defects around dental implants. GBR uses a covering barrier membrane to block soft tissue invasion. The need for GBR is determined by type and size of remaining bone wall. For example, when implants are placed immediately after tooth extraction, bone healing will be successfully achieved without GBR if all surrounding bony walls are intact. On the other hand, the necessity of GBR increases as loss of bony wall increases. Briefly, GBR should be performed in cases of large defect or loss of bony wall.

In accordance with the various embodiments of the present invention, resorbable and non-resorbable membranes for use as barriers in GBR, are provided, which are made from resorbable or non-resorbable materials/polymers. Additionally, the membranes also contain an effective amount of LPAA to inhibit bacterial growth. In contrast to surface mineralization strategies or metal-based antibacterial systems such as zinc phosphate, the present invention provides an intrafibrillarly mineralized ESM with structurally embedded apatite and polymer-mediated antibacterial action, thereby overcoming the limitations of conventional GBR membranes in both osteoinductivity and infection control.

Poly(acrylic acid) (PAA) is a polymer with the chemical formula (CH₂—CHCO₂H)ₙ. It is a derivative of acrylic acid (CH₂═CHCO₂H). In addition to the homopolymer, various copolymers, crosslinked polymers, and partially deprotonated derivatives are also known. In an aqueous solution at neutral pH, PAA behaves as an anionic polymer, meaning that many of its side chains lose protons and acquire a negative charge.

LPAA typically has a low weight-average molecular weight, which usually ranges from approximately 1,000 to 12,000, with an optimal range between 2,000 and 10,000. LPAA can induce intrafibrillar mineralization of ESM, enhancing its mechanical properties while preserving its porosity and pore size. In addition, LPAA prevents bacterial proliferation on the membrane surface, demonstrating its dual bioactive properties-mineralization and antibacterial effects. Although LPAA is widely used in industry, its antibacterial ability is first demonstrated in the context of ESM mineralization. The mineralized ESM mimics bone structure, containing both apatite and organic components from the membrane, and has shown osteogenic activities, making it a promising material for bone regeneration.

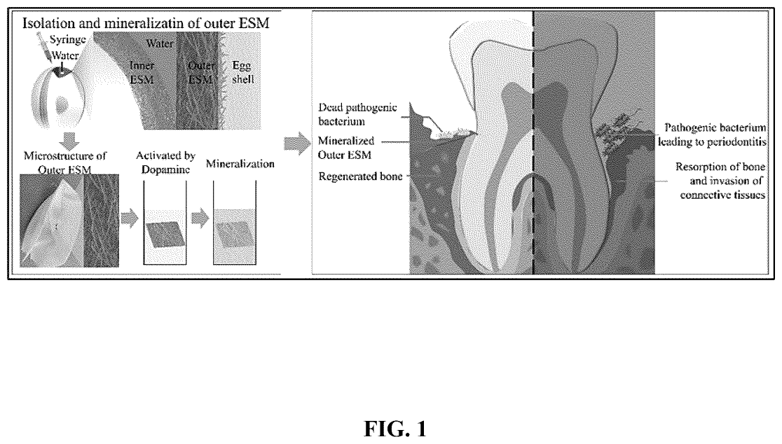

ESM is the by-product of the food processing industry which is considered as waste material. ESM is a unmineralized semi-permeable membrane consisting of two individual layers with fibrous meshwork structures between the egg albumin and the inner surface of the eggshell. (FIG. 1). The ESM possesses a layered, mesh-like, and fibrous proteinaceous microstructure that is highly insoluble but customizable. ESM is deposited as an interlaced fiber meshwork with three morphologically distinct layers: a thin limiting membrane (LM), an inner membrane (inner shell membrane (ISM)), and an outer-layer membrane (outer shell membrane (OSM)). The fibers of the outer and the inner membranes are interlaced throughout most of their surface but become separated at the air cell (broad end of the egg). Each fiber presents a similar construction, with a core rich in collagen, surrounded by a fuzzy glycoproteic mantle. However, the fiber position, orientation, and size differ for each membrane layer. The inner membrane is thinner than the outer membrane, being around 15-26 μm thick, with a smaller fiber width of 0.1 to 3 μm and a diameter of 1.5 to 2 μm. The outer membrane is around 50-70 μm thick, with fibers 1 to 7 μm in width and 2.5-5 μm in diameter. The fibers of the outer ESM penetrate the mammillary knobs of the shell, forming a bud-like structure that is partially calcified.

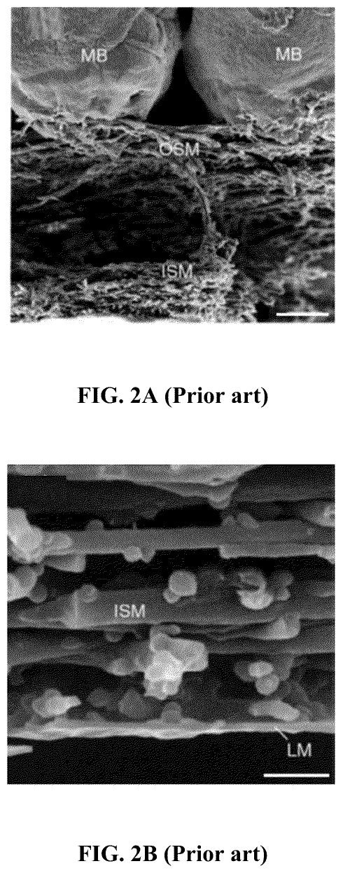

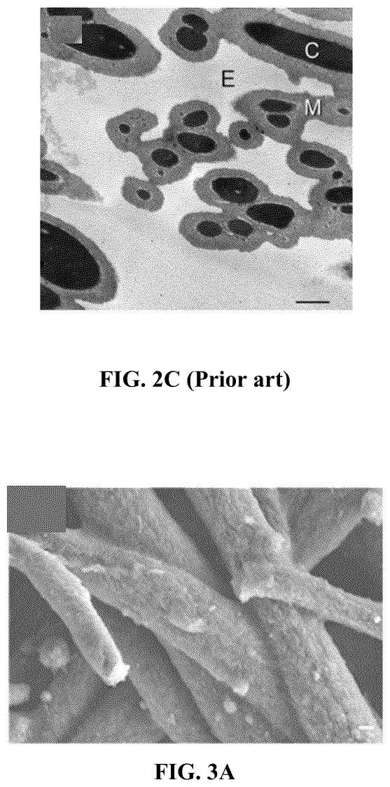

FIG. 2A (prior art) shows SEM micrograph of membrane and mammillary columns/bodies interface. It is possible to differentiate the inner shell membrane (ISM) from the outer shell membrane. The mammillary bodies (MB) are also marked. FIG. 2B (prior art) shows detail of the ISM and the LM. FIG. 2C (prior art) shows TEM micrograph of outer membrane fibers, depicting the highly electron-dense collagen-containing core (C) and the less electron-dense glycoproteic mantle (M), separated by extra-fiber spaces (E).

In one embodiment, the functionalized bioactive GBR material is a multi-component material, including an ESM which is functionalized by mineralization in the presence of LPAA. The ESM therefore includes at least one mineral deposited therein, preferably, intrafibrillar deposition, for example apatite and effective amounts of LPAA ((hereinafter, “mineralized LPAA-ESM”). The present materials can be applied to a subject in need thereof, for example, a subject with a buccal bone defect. In dome forms, the subject suffers from bone loss due to periodontal disease. Physical injury or pathological changes such as removal of a tumor can result in large bone defects in the buccal cavity.

The intrafibrillar mineralization of ESM is achieved by adding LPAA to the calcification solution, which not only eliminates bacteria but also promotes cell proliferation. This process endows the membrane with antibacterial properties by binding the antibacterial LPAA to the apatite, which is intrafibrillarly generated within the membrane fibers.

In one embodiment, the ESM component used in the present invention is an OSM of the ESM. The OSM has distinctive structures on its outer surface named mammillary knobs. These are discrete organic matter aggregations that function as nucleation sites for calcite. These sites possess a different protein composition from the rest of the fibrous membrane, containing a high concentration of globular proteins and proteoglycans. The outer membrane contains discrete aggregates of organic matter intermixed with the fibrillar material and embedded into the mammillary knobs, which, if seen from the mineral columns, resemble an “opening flower bud”, also referred to as “bud-like structures.” These structures, also known as “mammillary cores, calcium reserve assembly, or mammillae” from a mineralization point of view) have been described as having a base plate that contains amorphous calcium carbonate and a “calcium reserve body” which are calcium crystals embedded in an organic core rich in sulfated proteoglycans. These organic aggregates are nucleation centers where the transition from amorphous calcium carbonate to calcite occurs (Reviewed in Torres-Mansilla, et al., Polymers (Basel). 2023 Mar. 8; 15(6): 1342).

In one embodiment, the ESM is primarily composed of fibrous proteins such as collagen type I. Eggshell membranes may also contain glycosaminoglycans, such as dermatan sulfate, chondroitin sulfate, and sulfated glycoproteins including hexosamines, such as glucosamine. Other components identified in eggshell membranes are hyaluronic acid, sialic acid, desmosine, isodesmosine, ovotransferrin, lysyl oxidase, lysozyme, and β-N-acetylglucosaminidase.

In one embodiment, the ESM can be obtained from the avian members, such as chicken, ducks, or geese. Egg membranes from these sources are similar in terms of microstructures. In some forms, goose eggshell membranes are suitable as a GBR membrane candidate because of their thickness and higher mechanical strength.

The membrane can be separated from the eggshell using various methods, depending on the experimental requirements. These methods include chemical processes, mechanical techniques, steam treatment, and vacuum-assisted separation, each offering distinct advantages for different applications. The choice of separation method is determined by factors such as desired membrane integrity, scalability, and the specific properties needed for further processing.

In certain forms, the only fibrils and fibers present in the mineralized LPAA-ESM originate from the ESM itself. That is, the disclosed material does not contain fibers made from either absorbable or non-absorbable polymers, such as polytetrafluoroethylene (including expanded PTFE), poly(lactic acid), or copolymers like poly(lactic acid/glycolic acid), among others.

In one embodiment, the mineralized membrane has antibacterial activity. No live bacterium can be found on the membrane.

The bacterium may include Gram-positive species. Examples of Gram-positive species include Staphylococcus aureus, Streptococcus mutans, Streptococcus sanguinis, and Listeria monocytogenes.

In one embodiment, the mineralized membrane is safe for Sprague-Dawley bone marrow mesenchymal stem cells (SD-BMSCs).

In addition, the mineralized LPAA-ESM has improved mechanical properties, higher porosity, and larger pore size compared to a non-mineralized ESM.

The mineralized LPAA-ESM is preferably porous, have a median pore diameter of about 3650 nm-6760 nm; about 4160 nm-6240 nm; about 4680 nm-5720 nm; about 4940 nm-5460 nm; about 5145 nm-5250 nm as measured by mercury intrusion porosimetry (AutoPore IV 9500). In some forms, the median diameter is about 5198.51 nm.

In one embodiment, the LPAA-ESM has porosity of at least 40% and up to 70%. In some forms, the porosity is about 50% and up to 60%, for example about 51%, 52%, 53%, 54%, 55%, 56%, 57%, 58% or 59%.

In one embodiment, the average pore size of the material is about 285 nm-530 nm; about 324 nm-490 nm; about 365 nm-447 nm; about 385 nm-426 nm; about 402 nm-410 nm as measured by using the mercury injection capillary pressure (MICP) method. In some forms, the average pore size is about 405.98 nm.

In one embodiment, the average pore size of 1-day mineralized ESM is about 805 nm-1495 nm; about 920 nm-1380 nm; about 1035 nm-1265 nm; about 1093 nm-1208 nm; about 1139 nm-1162 nm as measured by using the MICP method. In some forms, the average pore size of 1-day mineralized ESM is about 1150.05 nm.

In one embodiment, the average pore size of 3-day mineralized ESM is about 282 nm-525 nm; about 323 nm-485 nm; about 364 nm-445 nm; about 383 nm-424 nm; about 399 nm-408 nm as measured by using the MICP method. In some forms, the average pore size of 3-day mineralized ESM is about 403.68 nm.

In one embodiment, the mineralized LPAA-ESM (samples of 1×3 cm²) has a Youngs's modulus of at least 100 MPa, preferably, up to 500 MPa. Therefore, the LPAA-ESM (samples of 1×3 cm²) can have a Youngs's modulus of about 200, 300, or 400 MPa.

In one embodiment, the mineralized membrane exhibits osteogenic activities as demonstrated in both in vitro and in vivo data.

Moreover, the present invention further provides a method of making mineralized LPAA-ESM which preferably contains intrafibrillar mineralization. The method includes contacting unminealized ESM with dopamine solution having a concentration of 2 mg/mL for 24 hours, immersing the dopamine treated ESM into a calcification solution (5.83 mmol/L CaCl₂·2H₂O, 3.5 mmol/L K₂HPO₄, 1.17 mmol/L NaF, and 135.7 mmol/L NaCl buffered with 10 wt % Tris and 1 mol/L hydrochloric acid at a pH value of 6.9.) containing LPAA at a concentration of about 9 mg/ml, 10 mg/ml, 11 mg/ml, 12 mg/ml, 13 mg/ml, 14 mg/ml, 15 mg/ml or 16 mg/ml, preferably about 12.5 mg/ml and incubating at 37° C. for mineralization, for at least 6 hrs, up to 3 days, preferably, between 12 and 24 hours.

In one embodiment, LPAA present in the mineralized membrane has anti-planktonic bacterial ability. The MIC of LPAA is 20 mg/mL, 5 mg/mL, 2.5 mg/mL, 2.5 mg/mL, 1.25 mg/mL, 1.25 mg/mL, and 1.25 mg/mL at the bacterial concentration of 1×10¹¹ CFU/mL, 1×10¹⁰ CFU/mL, 1×10⁹ CFU/mL, 1×10⁸ CFU/mL, 1×10⁷ CFU/mL, 1×10⁶ CFU/mL, and 1×10⁵ CFU/mL, respectively. The minimal bactericidal concentrations (MBC) of LPAA are 5 mg/mL, 2.5 mg/mL, 2.5 mg/mL, 2.5 mg/mL, 2.5 mg/mL, and 1.25 mg/mL at the concentration of 1×10¹⁰ CFU/mL, 1×10⁹ CFU/mL, 1×10⁸ CFU/mL, 1×10⁷ CFU/mL, 1×10⁶ CFU/mL, and 1×10⁵ CFU/mL, respectively.

In the various embodiments, the subject is a human person in need of guided bone regeneration, such as an individual with alveolar bone loss resulting from periodontal disease, trauma, surgical resection, or other maxillofacial defects. The material may be applied during dental implantation procedures, bone graft surgeries, or craniofacial reconstruction where preservation of bone volume, prevention of bacterial infection, and promotion of osteogenesis are desired.

In some embodiments, the composition is used to treat buccal bone injuries in a subject with hyperglycemia.

In one application of the present invention, the subject has a maxillofacial bone injury, such as one affecting the bone surrounding the teeth (where the teeth are intact and the surrounding area includes both periodontal and osseous tissues), with regeneration occurring in both the bone and tissue around the teeth.

In another application of the present invention, the subject to whom the guided bone regeneration material is administered may be a mammal. Suitable mammalian subjects include, but are not limited to, humans, non-human primates (e.g., monkeys, chimpanzees), canines (e.g., dogs), felines (e.g., cats), rodents (e.g., rats, mice), lagomorphs (e.g., rabbits), and swine.

The disclosed compositions and methods are based on the discovery of a method of making an intrafibrillar mineralized ESM that can be used in bone regeneration. The methods use LPAA to induce intrafibrillar mineralization of ESM and also, prevent the proliferation of bacteria on the membrane surface. In addition, the intrafibrillar mineralization improves the mechanical property of ESM and maintains the porosity and pore size of the membrane. Because of its dual bioactive property, LPAA is used for intrafibrillar mineralization of ESM and for antibacterial applications.

The present invention differs fundamentally from conventional apatite-containing GBR materials. While prior art often uses surface-deposited or premixed apatite that lacks biological integration, the disclosed method induces intrafibrillar mineralization within the collagen-rich matrix of the ESM. This results in aligned, nanoscale apatite crystals with lower crystallinity and higher solubility, enhancing calcium and phosphate ion release, which promotes osteogenic differentiation. Additionally, the use of LPAA confers intrinsic antibacterial properties to the membrane without relying on antibiotics or cytotoxic metals. This dual-function, biomimetic, structurally integrated GBR material addresses the shortcomings of existing systems by simultaneously providing mechanical support, osteoinduction, and antimicrobial action in a biocompatible form. The invention can be further understood in view of the following non-limiting examples which are specific embodiments of the disclosure.

### EXAMPLE

### Example 1—Acquisition of Outer ESM

The ESM, which is located between the eggshell and the egg white, includes outer and inner ESM. The outer eggshell membrane is rich in type I collagen, while the inner eggshell membrane is composed of type I and type V collagens. The fibers in inner ESM are densely arranged compared with outer ESM. Therefore, the inner ESM should be detached from the outer ESM. To obtain outer ESM, egg albumen and egg yolk were discarded, then an ingenious method inspired by maxillary sinus floor elevation was employed.

In detail, distilled water was continuously injected into the chamber between outer ESM and inner ESM by one syringe needle leading to the inner ESM being peeled off from the outer ESM by the pressure from distilled water. Afterwards, the eggshell combined with outer ESM was immersed into 17% ethylenediamine tetraacetic acid disodium salt (EDTA) solution. Four hours later, when the calcium carbonate embedded with ESM fiber was dissolved, the outer ESM was carefully peeled off from the eggshell and immersed into distilled water to remove the residual EDTA.

### Example 2—Mineralization of Outer ESM

In untreated ESM, cysteine in mantle structure as a mineralization inhibitor blocked the intrafibrillar mineralization of the fibers in the ESM. Cysteine is generated during the generation of ESM and exist naturally in mantle structures of ESM fibers. The existence of cysteine inhibits the intrafibrillar mineralization activities of ESM fibers. Therefore, when eggshell is generated in the body of an animal, deposition of calcium carbonate only happens on ESM surface but not in ESM fibers. During the eggshell forming process, amorphous calcium carbonate can only deposit on membrane surface but not in the fibers.

A small quantity of extrafibrillar deposition does not contribute to the mechanical property evaluation, while dense deposition is not conducive to nutrient transport and the brittleness of the densely deposited eggshell make it unlikely to be applied as a marginal adaptive GBR material. Therefore, dopamine and LPAA were applied to promote the intrafibrillar mineralization of outer ESM.

Outer ESM was first immersed into a dopamine solution with a concentration of 2 mg/mL for 24 hours. After being rinsed by distilled water for removal of residual dopamine, the dopamine treated outer ESM was immersed into a calcification solution with a concentration of 12.5 mg/mL LPAA (Sigma-Aldrich, St. Louis, MO, MW=3000).

Calcification solution is a mineralization medium containing 5.83 mmol/L CaCl₂·2H₂O, 3.5 mmol/L K₂HPO₄, 1.17 mmol/L NaF, and 135.7 mmol/L NaCl buffered with 10 wt % Tris and 1 mol/L hydrochloric acid at a pH value of 6.9. The outer ESM was incubated at 37° C. for mineralization. The calcification solution was refreshed every 24 hours. Samples mineralized for 1 and 3 days were rinsed three times by distilled water, stored in distilled water at 4° C.

### Example 3—Characterization of Mineralized Outer ESM

Unmineralized and mineralized outer ESM were rinsed with distilled water, dehydrated with graded ethanol and subjected to critical point drying (Quorum K850, England) before being coated with a thin layer (3 nm thickness) of pure gold onto their surface using the ion sputtering unit (operated at 22 mA for 30 sec) in a vacuum apparatus (SC D 050, Germany). Then these samples were characterized by scanning electron microscope (SEM) (Zeiss Gemini 500, Germany).

According to the SEM microphotograph, unmineralized and mineralized outer ESM were selected for TEM characterization. Before characterization, samples were immersed into acetone solution containing 15 wt % epoxy resin, after that the mixture was heated to 60° C. to remove acetone solvent, followed by drying in a vacuum oven. Afterward, 1 gram of ethidene diamine was used as curing agent and the mixture was stirred for 10 min before poured into polytetrafluoroethylene (PTEF) molds, which finally cured at room temperature for one day and postcured at 80° C. for 24 hours. Finally, samples embedded in epoxy resin were sliced into 300×500×0.07 μm ultrathin sections for TEM characterization.

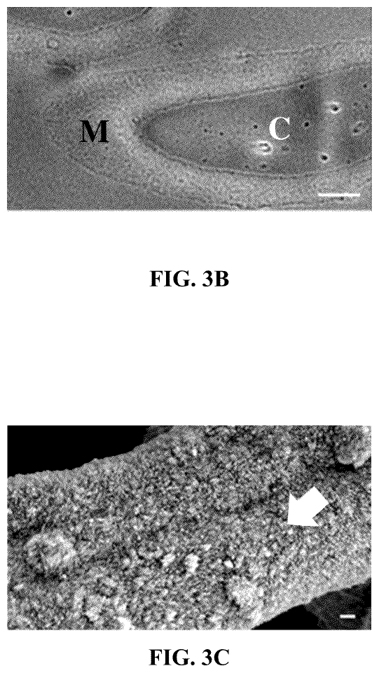

The SEM and TEM micrographs of the unmineralized outer ESM are shown in FIGS. 3A and 3B, respectively. The core fibers (marked C) and the mantle structure (marked M) of the unmineralized ESM fibers were clearly visible in FIG. 3B. Nanosized pores were observed in both the mantle and core structures. After immersing the membrane in a mineralization solution containing LPAA for 24 hours, SEM analysis of the membrane (FIG. 3C) and TEM images of the ultrathin section (FIG. 3D) indicated that intrafibrillar mineralization occurring on the first day. Densely packed apatite crystals (marked by white arrows in FIGS. 3C-3D) were observed in outer ESM fibers, with these crystals arranged in parallel or nearly parallel orientations along the fibers.

Extrafibrillar mineralization occurred, progressively covering the surface and blocking the micro-pores of the entire membrane as the mineralization period was extended. Although the calcium ions released from apatite deposition may promote osteogenic induction, this comes at the expense of reduced porosity and compromised marginal adaptation.

The extrafibrillar mineralization resulted from the prolonged treatment time. Since the fibers, which included both mantle and core structures, were porous and activated by dopamine, intrafibrillar mineralization initially took place within the fibers. Once intrafibrillar mineralization was completed, the extended mineralization time led to the formation of additional crystals on the fiber surface, resulting in extrafibrillar mineralization.

As the fibers were porous and activated by dopamine, apatite crystals preferentially deposited within the activated fibers, driving the intrafibrillar mineralization process in the beginning.

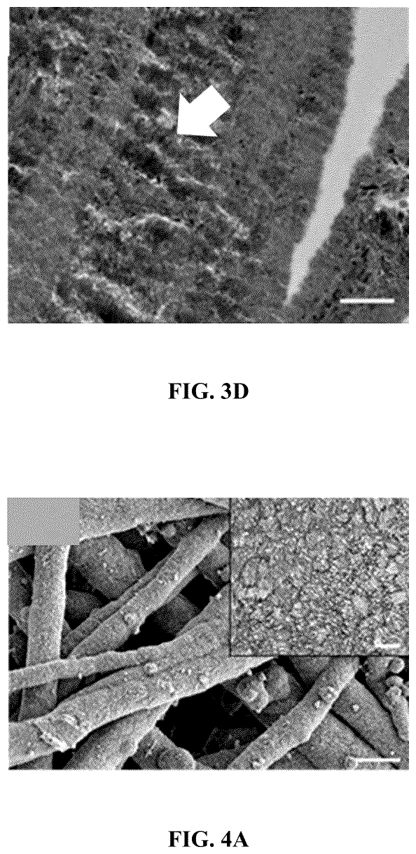

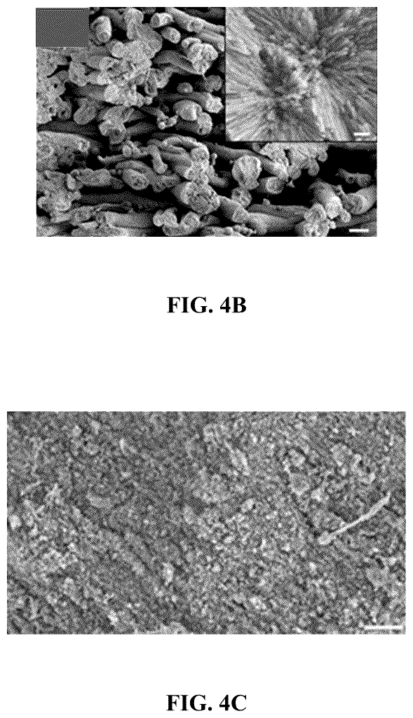

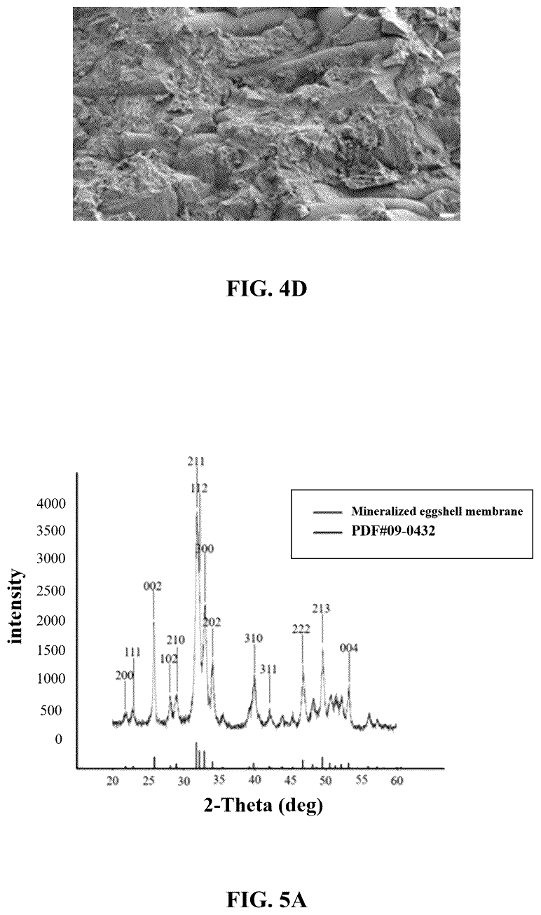

Compared to extrafibrillar mineralization (FIGS. 4C-4D), intrafibrillar mineralized membranes (FIGS. 4A-4B) may better mimic the microstructure of natural bone. The porosity of the membranes can be more effectively maintained by increasing the stiffness of the fibers, and this optimized porosity may enhance nutrient transport across the membranes. As a result, these microstructures could provide a more favorable environment for the proliferation and differentiation of osteogenic cells.

### Example 4—Physiochemical Properties of Intrafibrillar Mineralization of Outer ESM

To evaluate the impact of intrafibrillar versus extrafibrillar mineralization on porosity and mechanical properties, the physiochemical properties were further assessed.

X-ray diffraction (XRD) was further used to characterize the composition of the mineralized outer ESM. The membranes were analyzed by XRD (Rigaku TTR-III) under Cu-Kα radiation (λCu-Kα=0.1541841 nm, radiation at 40 kV and 200 mA) over the 2θ range of 20° to 60° (time per step: 0.15 s and step size: 0.02° s⁻¹). The obtained diffraction spectrum was compared with the documented XRD patterns of fluorapatite (FAP) (PDF #09-0432). FAP is an apatite. If there is no fluoride ion in the mineralization medium, the component of the deposition will be hydroxyapatite. To control the size of crystals, NaF is added in the mineralization solution. Therefore, part of hydroxyl groups in crystals are replaced by fluorion and FAP is generated.

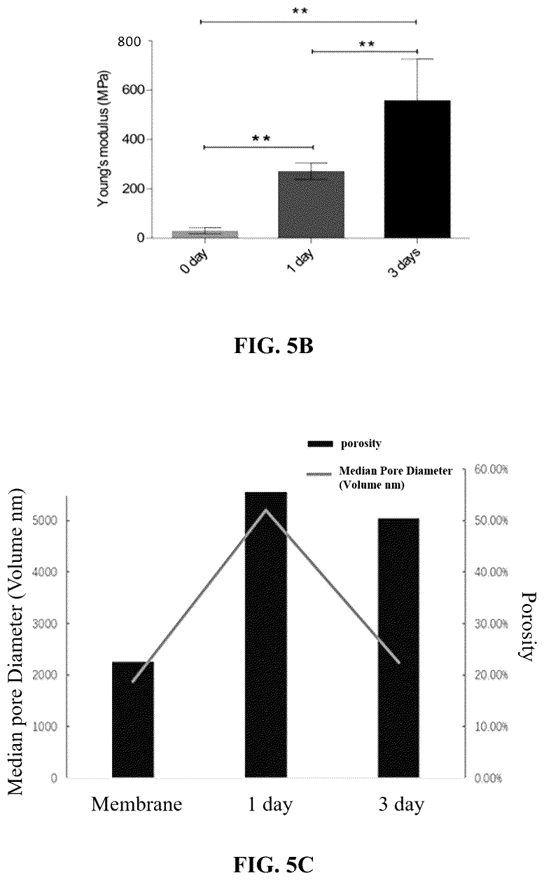

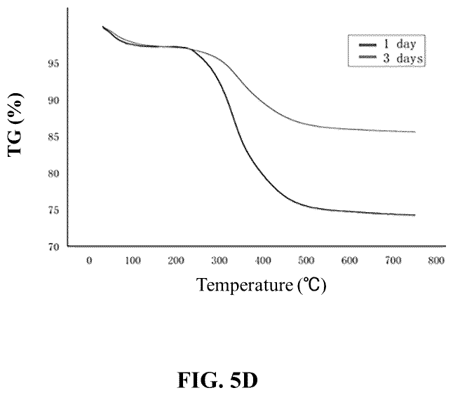

XRD was done on the intrafibrillar mineralized ESM fibers. XRD spectra of the crystals in FIG. 5A showed diffraction peaks of (002) at 2θ=25.8°, (211) at 2θ=31.8°, (112) at 2θ=32.2°, (300) at 2θ=32.9° and (004) at 2θ=53.1°. The spectrum matches with the standard peaks of FAP (Ca₅(PO₄)₃(OHF)) (JCPDS No. 09-0432). This indicated that the main component of the mineral deposition in the fibers is FAP.

### Example 5—Mechanical Properties of Unmineralized and Mineralized Outer ESM

The measurements of samples in each group were replicated three times (n=3). Before testing, the thickness of samples was recorded, samples of 1×3 cm² were clamped to the fixtures of mechanical apparatus. The samples were stretched to tensile failure at a rate of 10 mm/min. The data were recorded using the built-in software. The yield stress was calculated by dividing the maximum load by the cross-sectional area of the samples. The failure strain was calculated by dividing the change in length by the initial length of specimen. The stress-strain graph was produced by using Origin Pro 9.0 (Wellesley Hills, MA) and the elastic modulus was calculated as the gradient of the elastic region of the curve (2=stress/strain).

The mechanical properties of ESM and mineralized ESM were illustrated in FIG. 5B. There is significant difference in the values of Young's modulus between mineralized ESM and unmineralized ESM (p<0.05), as well as between ESM mineralized for one day and ESM mineralized for three days. With the extension of mineralization time, Young's modulus of ESM also increased gradually. This may be caused by a higher mass ratio of the deposited inorganic FAP. It should be noted that a higher Young's modulus of ESM does not mean that the ESM had a greater potential to be developed into a GBR material. Although appropriately increased Young's modulus of ESM fibers may play a positive role in maintaining bone regeneration space and ESM porosity, increased stiffness of materials caused by excessive mineralization may cause marginal adaptation problems in ESM as a GBR material. Young's modulus of ESM can have a range of about 140 MPa-260 MPa or 150 MPa-250 MPa or 160 MPa-240M Pa or 170 MPa-230 MPa or 180 MPa-220 MPa, or 190 MPa-210 MPa or 195 MPa-205 MPa or more preferably 200 MPa.

To study the effect of mineralization process on the outer ESM porosity, membranes prior and after mineralization were characterized by the MICP method.

Referring to FIG. 5C, the porosity and the median pore diameter did not increase with the extension of mineralization time. ESM mineralized for 1 day had higher porosity than unmineralized ESM and ESM mineralized for 3 days. On the one hand, one day's mineralized on ESM may improve the porosity of ESM by increasing the mechanical strength of fibers and thus preventing the collapse of fibers. On the other hand, three days' mineralized on ESM may cause extrafibrillar deposition of FAP crystals on fiber surface, thus lead to a decrease in porosity of the membrane. Therefore, only appropriately mineralized ESM has higher porosity. An ideal size of pores should be as big as possible for nutrient transporting but should be smaller than cells so that the membrane can prevent the invasion of some cells like fibroblast. The size of fibroblast is larger than 10 micrometers. When the pore size is less than 10 micrometers, membrane with a larger size will be more effective in nutrients transport. Therefore, mineralized membrane with a pore size of 5,198.51 nm is considered suitable candidate among all the membranes.

For the ratio of apatite in the mineralized membrane at different stages, thermogravimetric analysis was conducted, unmineralized and mineralized outer ESM were dried at 70° C. for 16 hours before testing. Thermogravimetric analysis (TGA, Q5000IR, USA) was performed under atmospheric oxygen to quantify the amount of organic content in the mineralized and unmineralized membrane respectively. The temperature was raised from room temperature to 800° C. based on a heating rate of 10° C.

Referring to FIG. 5D, the inorganic content was measured by thermogravimetric analysis. Inorganic content of the mineralized ESM increased with the increasement of mineralization time. This trend explained the differences in mechanical properties, as well as porosities in FIGS. 5B-5C.

### Example 6—Antibacterial Properties of LPAA

37 grams of brain-heart infusion broth (BHI, Difco Laboratories, Detroit, USA) was dissolved in 1000 ml distilled water and sterilized at 121° C. 40 mg/mL LPAA (MW=3000) was added into the BHI solution and the pH value of the solution was adjusted to 7 by NaOH. Two-fold serial dilutions of the BHI solution (LPAA concentrations from 40 mg/mL to 1250 μg/mL) were pipetted into 96-well cell culture plates (100 μL per well). Then 100 μL of bacterial suspension with final density of S. aureus from 10¹¹ to 10⁵ CFU/mL was pipetted into each well and cultured with each medium at 37° C.

24 hours later, absorbance of BHI solution containing S. aureus was measured. The minimal inhibitory concentration (MIC) of LPAA was defined as the lowest concentration that caused at least a 90% reduction in absorbance compared with the negative control group.

Meanwhile, S. aureus in each group was suspended by sonication for 30 seconds after being co-cultured with 100 μL of the BHI solution containing different concentrations of LPAA for 24 hours. Ten-fold serial dilutions of the suspensions were plated in duplicate on agar plates and incubated for 48 hours. Colony forming unit (CFU) counting was processed afterwards. The minimal bactericidal concentration (MBC) was defined as the lowest concentration of LPAA resulting in no colony formation on agar plates after incubation.

### Example 7—Antibacterial Properties of Mineralized Outer ESM

3 unmineralized ESM (10 mm in diameter) and 3 mineralized ESM (lasted for 1 day, 10 mm in diameter) were autoclave-sterilized prior to the antibacterial activity testing, then placed in 12-well plates. 20 μL BHI containing 10⁵ CFU/mL of S. aureus was seeded on sample surfaces and cultured for 4 hours prior to adding 2 mL of BHI in each well. The membranes were incubated for 24 hours at 37° C. After incubation, the membranes were washed once with PBS. One randomly selected membrane in each group was stained using the LIVE/DEAD BacLight Bacterial Viability Kit (L7012, Thermo Fisher Scientific, Waltham) in the dark for 30 minutes. Fluorescence images were obtained using CLSM. One randomly selected membrane in each group was dried by critical point drying (Quorum K850, England) and observed by SEM.

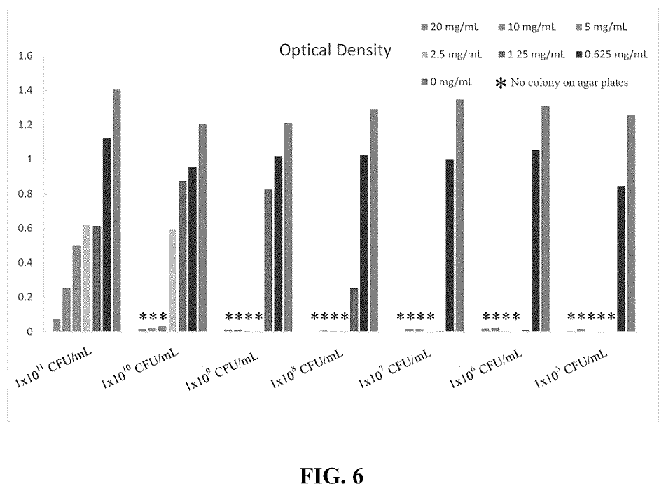

The MIC of LPAA at the S. aureus concentration from 10⁵ CFU/mL to 10¹¹ CFU/mL was 1.25, 1.25, 1.25, 2.5, 2.5, 5, and 5 mg/mL, respectively while the MBC from 10⁵ CFU/mL to 10¹⁰ CFU/mL was 1.25, 2.5, 2.5, 2.5, 2.5, and 5 mg/mL (FIG. 6). It can be concluded that LPAA has strong antibacterial properties on S. aureus, and higher concentrations of LPAA had stronger inhibitions on the proliferation of S. aureus.

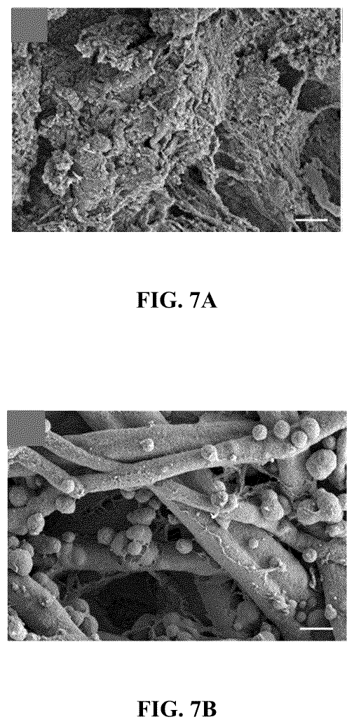

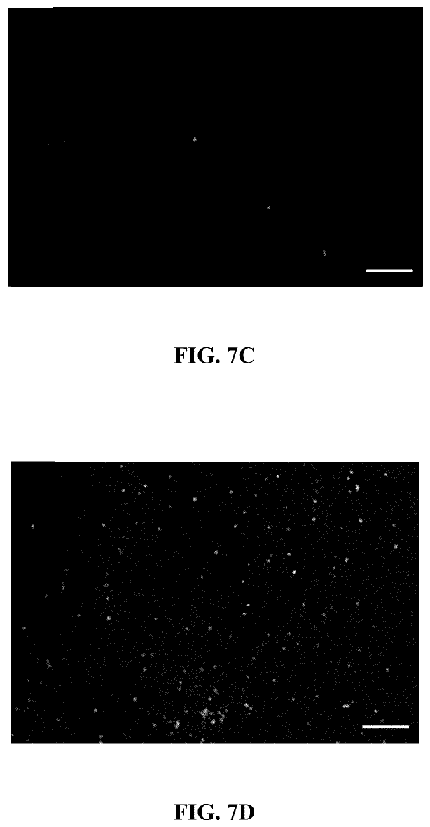

Differences in antibacterial properties between unmineralized and mineralized ESM were obvious as shown in FIGS. 7A-7D. No colony was observed on the mineralized ESM surface as shown in FIGS. 7A-7C while a colony was obviously observed on the unmineralized ESM surface as shown in FIGS. 7B-7D. This indicated that mineralized ESM has antibacterial properties.

### Example 8—Biocompatibility Evaluation of Intrafibrillar Mineralized Outer ESM

3 unmineralized ESM (10 mm in diameter) and 3 mineralized ESM (lasted for 1 day, 10 mm in diameter) were autoclave-sterilized prior to biocompatibility evaluation and then placed in 12-well plates. 3 days after the seeding of 10,000 cells on each sample surface, one random sample in each well were washed with PBS and stained by 4′,6-diamidino-2-phenylindole (DAPI) and phalloidine. Besides, one randomly selected membrane in each group was dried by critical point drying (Quorum K850, England) and observed by SEM for cell morphology observation.

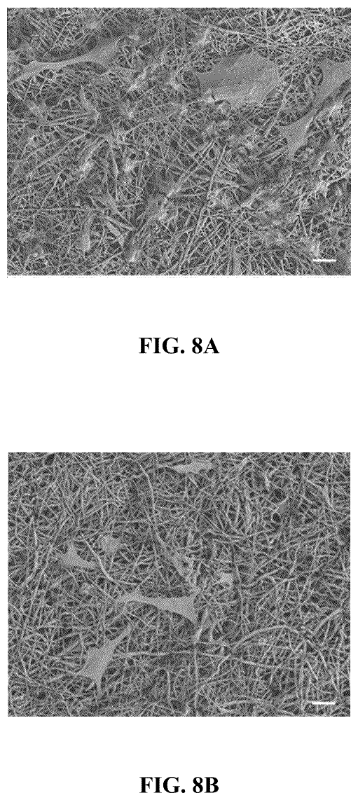

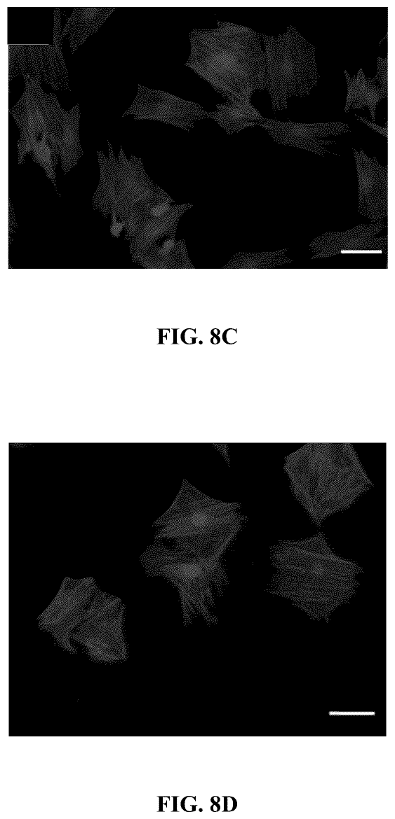

SD-BMSCs attached and stretched well on the surface of unmineralized ESM and intrafibrillar mineralized ESM. In FIGS. 8A-8D, the SEM micrograph and the fluorescence staining micrograph all indicated that the mineralization process did not has adverse effects on biocompatibilities.

### Example 9—In Vitro Osteogenic Activities Evaluation of Mineralized Outer ESM

The osteogenic potential of mineralized outer ESM was evaluated using two distinct experimental protocols to assess its ability to induce both ectopic and in situ osteogenesis.

### Ectopic Osteogenesis Evaluation:

In the ectopic osteogenesis model, 1×10⁴ BMSCs were added into each well plate and replaced the culture medium with osteogenic induction medium including 10⁻⁸ mmol/L dexamethasone, 1×10⁻² mol/L sodium β-glycerophosphate, 5×10⁻⁵ mol/L ascorbic acid, and 10% fetal bovine serum after the cells were fully adhered to the well plate's surface 24 hours later. The experimental group used mineralized outer ESM, while commercial Bio-Gide® and unmineralized outer ESM were used as control groups. A Transwell system was used to isolate the membranes from the cells, allowing only the compounds released from the membranes to penetrate through the small pores of the Transwell into the culture medium.

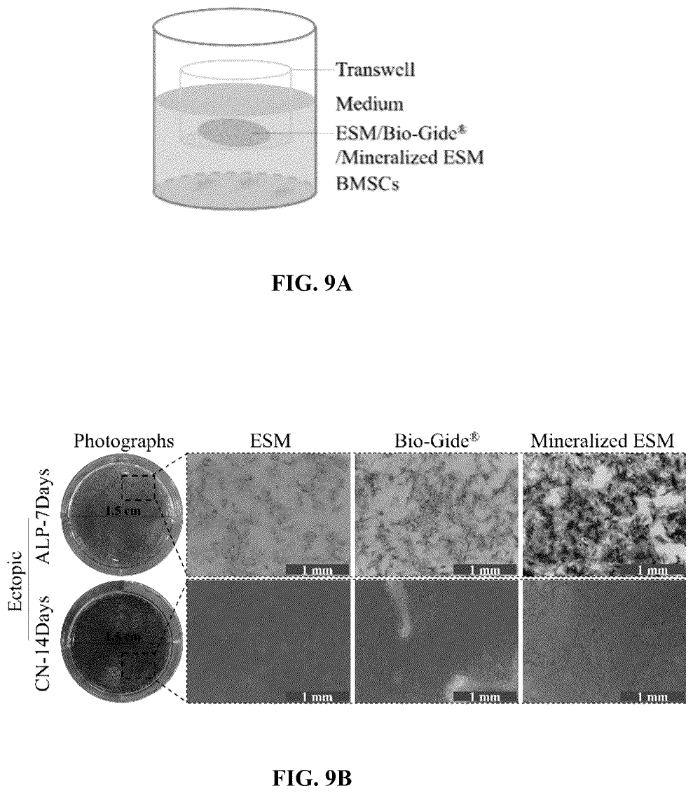

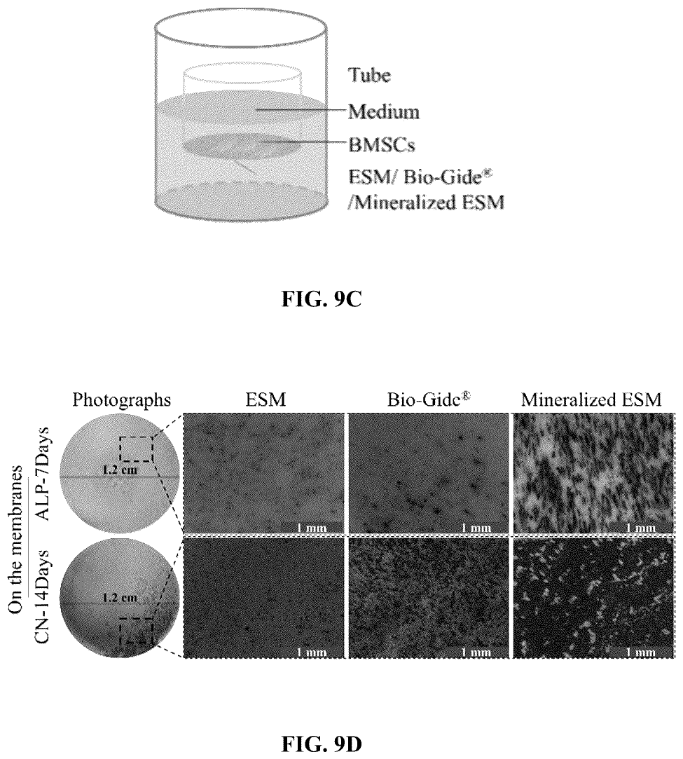

After 7 days of culture, the induction of ALP was evaluated. As shown in FIG. 9B, mineralized outer ESM induced the most prominent ALP expression in BMSCs on the seventh day, indicating early osteogenic activity. In contrast, neither the unmineralized outer ESM nor Bio-Gide® membranes exhibited any noticeable osteogenic induction at this time. After 14 days of culture, no obvious calcium nodule (CN) formation was observed in any of the groups, and the cells were disorganized, likely due to the inappropriate substrate for long-term cell culture.

### In Situ Osteogenesis Evaluation:

To evaluate the potential of mineralized outer ESM to induce in situ osteogenesis, a novel bone defect model was designed. A 12-mm-diameter, short, hollow polypropylene (PP) tube was closed at one end with the membrane, with the open end positioned upwards in a sterile 15-mm-diameter culture plate. After dropping the cell suspension into the tube, the cells spontaneously adhered to the membrane's surface, preventing the exchange of nutrients and waste between the cells and the external medium. This setup simulated a closed bone defect environment, with the membrane acting as a guided bone regeneration barrier.

The same staining and culture procedures were applied as in the ectopic model. On day 14, as shown in FIG. 9D, mineralized outer ESM induced significant ALP expression and an extensive amount of CN formation, demonstrating strong osteogenic differentiation in situ. This suggests that mineralized outer ESM promotes in situ osteogenesis more effectively than the unmineralized outer ESM and Bio-Gide® controls. The surface of the mineralized outer ESM membrane also appeared to support better osteogenic expression and more homogeneous cell distribution compared to the other groups, as evidenced by the staining results shown in FIG. 9D in comparison to FIG. 9B.

Based on these findings, it can be concluded that mineralized outer ESM has a positive impact on both ectopic osteogenesis and in situ osteogenesis, potentially promoting bone healing more effectively than the control groups.

### Example 10—In Vivo Osteogenic Activities Evaluation of Mineralized Outer ESM

To evaluate the in vivo osteogenic properties of mineralized outer ESM, a study was conducted using 24 New Zealand rabbits (age: 90-120 days; body weight: 2.1 kg). All procedures were performed in compliance with the National Institutes of Health guidelines for the care and use of laboratory animals.

The rabbits were anesthetized with isoflurane gas inhalation (Isoba Vet, Schering-Plough, Uxbridge, UK), and anesthesia was maintained throughout the procedure. The surgical site was shaved and cleaned with 0.5% iodophor. A circular critical-size defect (CSD) of 8 mm internal diameter was created on the bilateral femoral epiphysis (trabecular bone region), with generous irrigation using 0.9% NaCl. The full thickness of both the cortical and trabecular bone was removed. The defects were then covered with various membrane types according to the experimental groups: unmineralized outer ESM or Bio-Gide® membrane was used for the negative and positive control groups, respectively, while the experimental group was covered with mineralized outer ESM. The defects left uncovered with any membrane served as the blank control group. After the surgery, the subcutaneous and skin layers were closed with non-resorbable sutures. The animals were sacrificed at three different time points: 2 weeks, 4 weeks, and 3 months post-surgery, using an overdose of barbiturate (Mebumal, ACO Lakemedel AB, Solna, Sweden). The skin was carefully reopened, and the bone tissue along with the overlying membrane and soft tissues were harvested and preserved in formalin for 24 hours.

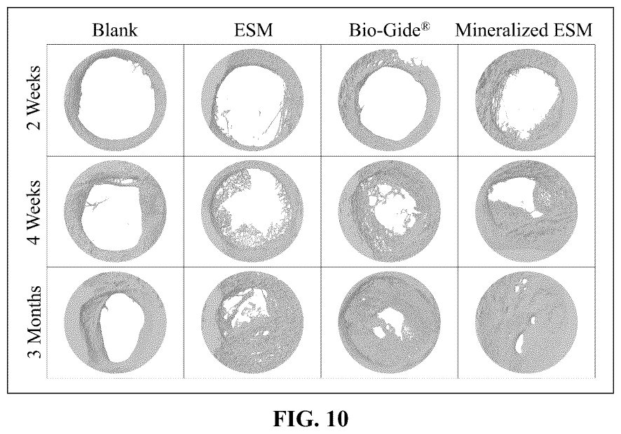

Three-dimensional (3D) reconstruction of the bone defect area was obtained by scanning the samples using micro-CT (μCT 80; Scanco Medical, Switzerland). As shown in FIG. 10, the results indicated that mineralized outer ESM induced osteogenesis earlier than the other groups. This early osteogenesis is likely attributed to the release of calcium ions and phosphate groups from the mineralized membrane. Furthermore, the experimental group demonstrated a significantly higher rate of defect area closure by the third month compared to the control groups.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

1. A guided bone regeneration material, comprising:
a mineralized eggshell membrane (ESM) comprising fibers and fibrils;
intrafibrillarly deposited apatite; and
low-molecular-weight polyacrylic acid (LPAA) bounded to the intrafibrillarly deposited apatite,
wherein the intrafibrillarly deposited apatite comprises aligned, densely packed apatite crystals formed within internal structures of the fibers and fibrils, such that a mineral phase is embedded inside a fibrillar matrix instead of externally coated on to the ESM, and is guided by the presence of LPAA; and
wherein the guided bone regeneration material has a porosity of approximately 40% to 70%.

2. The guided bone regeneration material of claim 1, wherein the ESM is an outer shell membrane comprising mammillary knobs.

3. The guided bone regeneration material of claim 1, wherein the apatite crystals of the intrafibrillarly deposited apatite are fluorapatite having X-ray diffraction peaks at 2θ angles of approximately 25.8°, 31.8°, 32.2°, 32.9°, and 53.1°.

4. The guided bone regeneration material of claim 1, wherein the LPAA has an average molecular weight of 1,000-20,000 Da.

5. The guided bone regeneration material of claim 1, wherein the LPAA is present in an amount effective to inhibit bacterium growth.

6. The guided bone regeneration material of claim 5, wherein the bacterium comprises Staphylococcus aureus, Streptococcus mutans.

7. The guided bone regeneration material of claim 1, wherein the guided bone regeneration material has an average pore size between 250 nm to 1500 nm.

8. The guided bone regeneration material of claim 7, wherein the ESM is mineralized for one day and has an average pore size between 800 nm to 1500 nm.

9. The guided bone regeneration material of claim 7, wherein the ESM is mineralized for three days and has an average pore size of between 300 nm to 600 nm.

10. The guided bone regeneration material of claim 1, wherein the guided bone regeneration material has a Young's modulus of at least about 100 MPa and up to about 500 MPa.

11. The guided bone regeneration material of claim 1, wherein the fibers and fibrils are derived solely from the ESM and are free of exogenous polymeric fibers.

12. The guided bone regeneration material of claim 1, wherein the guided bone regeneration material is biocompatible with Sprague-Dawley bone marrow mesenchymal stem cells (SD-BMSCs).

13. The guided bone regeneration material of claim 1, wherein the guided bone regeneration material promotes ectopic and in situ osteogenesis.

14. A method of preparing the guided bone regeneration material of claim 1, comprising:
contacting an unmineralized ESM with a dopamine solution at a concentration of about 1 to 5 mg/mL for 24 hours to form a dopamine-treated ESM;
immersing the dopamine-treated ESM in a calcification solution comprising LPAA at a concentration of about 10 to 15 mg/mL; and
incubating at 37° C. for 12 to 24 hours to induce intrafibrillar mineralization to form the guided bone regeneration material;
wherein the LPAA guides formation of aligned, densely packed apatite crystals embedded within an internal fibrillar matrix; and
wherein the guided bone regeneration material exhibits improved Young's modulus and porosity relative to the unmineralized ESM.

15. The method of claim 14, wherein the calcification solution comprises 5.83 mmol/L CaCl₂·2H₂O, 3.5 mmol/L K₂HPO₄, 1.17 mmol/L NaF and 135.7 mmol/L NaCl, buffered with 10 wt % Tris and 1 mol/L HCl at pH 6.9.

16. The method of claim 14, wherein incubation is performed at 37° C. for at least 6 hours and less than 72 hours.

17. A method for promoting bone regeneration in a subject in need thereof, comprising applying the guided bone regeneration material of claim 1 to a bone defect site in the subject.

18. The method of claim 17, wherein the subject suffers from periodontal disease in need of bone regeneration.

19. The method of claim 17, wherein the subject suffers from hyperglycemia or impaired wound healing.

20. The method of claim 17, wherein the guided bone regeneration material induces early-stage osteogenesis within 7 days and promotes defect closure within 3 months.
